(19) United States
(12) Patent Application Publication
(10) Pub. No.: US 2014/0113821 A1
(43) Pub. Date: Apr. 24, 2014

(54) POLYSACCHARIDE-BASED HYDROGEL POLYMER AND USES THEREOF

(71) Applicants: Frank Gu, Kitchener (CA); Mohit Singh Verma, Brampton (CA); Drew William Davidson, Orangeville (CA); Benjamin Charles Lehtovaara, Georgetown (CA)

(72) Inventors: Frank Gu, Kitchener (CA); Mohit Singh Verma, Brampton (CA); Drew William Davidson, Orangeville (CA); Benjamin Charles Lehtovaara, Georgetown (CA)

(21) Appl. No.: 14/123,480

(22) PCT Filed: Jun. 1, 2012
(86) PCT No.: PCT/CA2012/050369

§ 371 Date: Dec. 2, 2013

Related U.S. Application Data
(60) Provisional application No. 61/492,995, filed on Jun. 3, 2011.

(51) Int. Cl. A01N 25/10; A61K 47/36; C05G 3/00

(52) U.S. Cl. CPC A01N 25/10; C05G 3/0029; A61K 47/36
USPC 504/197; 504/359; 71/11; 71/23; 424/400; 424/184.1; 424/405

(57) ABSTRACT

A method of preparing a hydrogel for delivery of an active agent. The method includes providing an aqueous solution that includes the active agent; dispersing or dissolving a gel-forming polymer in the aqueous solution to form a polymer solution; and cross-linking the polymer in the polymer solution to form the hydrogel which encapsulates the active agent.

### CROSS-REFERENCE

This application claims the benefit of priority of U.S. Provisional Application No. 61/492,995, filed Jun. 3, 2011. The content of the provisional application is hereby incorporated by reference in its entirety.

### TECHNICAL FIELD

The present disclosure relates generally to a polysaccharide-based hydrogel and use thereof in agricultural as well as healthcare industry. More particularly, the present disclosure relates to a polysaccharide-based hydrogel polymer and uses thereof in the delivery of active agents to plants, animals or humans.

### BACKGROUND

Polymeric hydrogels have been utilized to retain moisture in soil around plant roots, as described in U.S. Pat. No. 5,185,024. These hydrogels can be applied in a bulk form or sprayed on the plants. Hydrogels have also been used to provide micronutrients such as iron sulfate to plants (U.S. Pat. No. 5,632,799). Such a formulation provides a sustained supply of micronutrients over several days. In order to protect plants from animals, formulations have been invented that incorporate plant derived toxins delivered in various forms (U.S. Pat. No. 7,052,708 B2). These formulations deter animals from disturbing vegetation without causing permanent damage to either the plant or the animal. These hydrogel systems typically disintegrate quickly in the soil or have a tendency to swell to volumes that can disturb plant growth.

Beta-glucans have presented pharmacological activity by stimulating immune response which in turn has applications in anti-tumor activity, wound healing as well as infection resistance (Chihara, 1970; Sasaki, 1978; Ohno, 2001; Yano, 1991; Wei, 2002; Portera, 1997;). Some examples of uses of beta-glucans include the formulation for constipation-relieving drug (Pub. No. US2005/0272694 A1), inclusion of pharmaceutically active botanical extract (Pub. No. US2006/0121131 A1), glucan for cancer therapy (Pub. No. US2006/0160766 A1), glucan for skin application (Pub. No. US2007/0224148 A1), medical application of glucans in animals (Pub. No. US2010/0267661 A1) and for the prevention of osteoporosis (U.S. Pat. No. 7,671,039 B2). These examples highlight the breadth of medical applications related to beta-glucans.

Curdlan is a water insoluble, linear, high molecular weight beta-1,3-glucan biosynthesized by the soil bacterium Alcaligenes Faecalis var. Myxogenes, as well as Agrobacterium biobar. Curdlan has been studied extensively in the literature for its helix forming capacity and ability to gel when heated to form resilient gels (Harada, 1979; Deslandes, 1980). In addition, Curdlan has been investigated for its ability to impart increased immunocompetency to an applicable host (Sasaki, 1978; Sonck, 2010).

The structure forming properties of Curdlan have led to a number of applications in food science as a thermal structural hydrogel (Nakao, 1991; Funami, 1998) and other similar Beta-1,3-glucans have been used as scaffolds in nanostructure formation (Dunstan, 2007; Haraguchi, 2005). In the recent literature, the ability to form liquid crystal gels when Curdlan is dialyzed against aqueous calcium chloride has been of ongoing interest (Dobashi, 2004; Nobe, 2005) with the hydrogel system being used to model the formation of similar gels formed from DNA (Furusawa, 2007; Dobashi, 2007).

The pharmacological potential of Curdlan has led to a number of applications in human drug delivery including the thermal gelation of Curdlan to encapsulate and release drugs (Kanke, 1995) and, recently, the use of pure Curdlan and its water soluble carboxymethylated derivative to coat nanoparticle systems encapsulating chemotherapeutic drugs has become an effective approach (Na, 2000; Kim, 2005; Subedi, 2009; Li, 2010). Curdlan and other beta-1,3-glucans have also been used to form water-soluble helical complexes (Kimura, 2000; Miyoshi, 2005) with appropriate modification to the Curdlan backbone necessary for complex formation (Koumoto, 2001; Hasegawa, 2007). The helical complexes formed with all 1,3-Beta-glucans require the presence of a homogenous nucleic acid, leading the most recent research to cause complex formation by appending a useful oligonucleotide with a section of homogenous nucleotides to facilitate complex formation (Karinaga, 2005). Appending the 1,3-Beta-glucan with water stabilizers such as poly(ethylene glycol) has allowed for improved cellular uptake and reduction in lysosomal degradation (Karinaga, 2006).

Deoxyribonucleic acid (DNA) has also been utilized to exemplify pharmacological activity. Particularly, CpG DNA is important for immunostimulatory applications such as vaccination as demonstrated previously (U.S. Pat. No. 7,749,979 B2). Other forms of DNA such as plasmid have also been exploited for their use in vaccines for atherosclerosis (U.S. Pat. No. 6,284,533 B1). While studying DNA, it was observed that a crystalline layer around DNA can provide protection from external degradation sources (Wolf, 1999).

Cellulose is a water insoluble polysaccharide composed of β(1→4) linked D-glucose units. The polymer backbone can be modified to change its solubility in water. One such derivative is carboxymethyl cellulose (CMC), which is water soluble. CMC can be used for formation of stable gel compounds by the use of ionic gelation as exemplified previously (U.S. Pat. No. 4,618,491). Cellulose and its derivatives have also been explored for ocular drug delivery, as discussed in US Patent Publication No. US 2011/0129516 A1, and for oral drug administration, as discussed in US Patent Publication US 2008/0226705 A1.

A delivery system is required that can be applied to plants, animals as well as humans for carrying various active ingredients such as water, crop protection agents, therapeutic agents, nucleic acids, while providing a controlled release of these agents.

### SUMMARY

In a first aspect, the present disclosure provides a polysaccharide-based polymer hydrogel that includes: a polysaccharide polymer; a cross-linker interacting with the polysaccharide polymer to cross-link the polysaccharide polymer; and an active agent encapsulated by the cross-linked polysaccharide polymer.

The cross-linker may be an ion, such as a metal cation, that interacts with counter-ion functional groups on the polysaccharide polymer.

The cross-linker may be a chemical cross-linker reacted with the polysaccharide polymer. The chemical cross-linker may be a multifunctional aldehyde, a multifunctional carboxylic acid, a multifunctional amine, a multifunctional amide, or a multifunctional isocyanate. In specific examples, the chemical cross-linker may be glutaraldehyde, succindialdehyde, citric acid, maleic acid, itaconic acid, tetramethylolacetylenediurea or toluene diisocyanate.

The active agent may be a small molecule; an immunostimulator; an anti-cancer molecule; a vaccine; a biopolymer; a crop protecting agent; or any combination thereof. In specific examples, the active agent is a plant fertilizer.

The polysaccharide polymer may be a peptidoglycan polymer.

The polysaccharide polymer may alternatively be a beta-glucan polymer or an alpha-glucan polymer. The alpha-glucan polymer may be an alpha-1,6-glucan with alpha-1,3 branches. The alpha-1,6-glucan with alpha-1,3 branches polymer may be dextran or polyaldehyde dextran.

The alpha-glucan polymer may alternatively be an alpha-1,4-; alpha-1,6-glucan. The alpha-1,4-; alpha-1,6-glucan polymer may be pullulan or starch.

The beta-glucan polymer may be a beta-1,3-glucan or a beta-1,4-glucan polymer.

The beta-1,3-glucan may be a beta-1,3-glucan with beta-1,6 branches, such as: schizophyllan, lentinan, pachyman, pachymaran, scleroglucan, grifolan or a chemical derivative thereof. The chemical derivative may be carboxymethylpachymaran, hydroxymethyl pachymaran, or hydroxypropyl pachymaran. Alternatively, the beta-1,3-glucan polysaccharide polymer may be a curdlan polymer or a carboxymethyl curdlan polymer.

The beta-1,4-glucan polymer may be a cellulose polymer, such as a carboxymethyl cellulose polymer, chitin or a chitin derivative.

The polysaccharide-based polymer hydrogel may additionally include an excipient. The excipient may be a bulking agent.

In another aspect, there is provided an ionically cross-linked polysaccharide-based polymer hydrogel for triggered delivery of an active agent encapsulated by the hydrogel. The hydrogel includes: a polysaccharide polymer; ions interacting with counter-ion functional groups on the polysaccharide polymer to cross-link the polysaccharide polymer; and an active agent encapsulated by the cross-linked polysaccharide polymer.

The delivery of the encapsulated agent may be triggered by a chelating agent that interacts with at least a portion of the ions and prevents them from cross-linking the polysaccharide polymer.

The active agent may be water; a small molecule; an immunostimulator; an anti-cancer molecule; a vaccine; a biopolymer; a crop protecting agent; or any combination thereof. In specific examples, the active agent is a water or a plant fertilizer.

The polysaccharide polymer may be a peptidoglycan polymer.

The polysaccharide polymer may alternatively be a beta-glucan polymer or an alpha-glucan polymer. The alpha-glucan polymer may be an alpha-1,6-glucan with alpha-1,3 branches. The alpha-1,6-glucan with alpha-1,3 branches polymer may be dextran or polyaldehyde dextran.

The alpha-glucan polymer may alternatively be an alpha-1,4-; alpha-1,6-glucan. The alpha-1,4-; alpha-1,6-glucan polymer may be pullulan or starch.

The beta-glucan polymer may be a beta-1,3-glucan or a beta-1,4-glucan polymer.

The beta-1,3-glucan may be a beta-1,3-glucan with beta-1,6 branches, such as: schizophyllan, lentinan, pachyman, pachymaran, scleroglucan, grifolan or a chemical derivative thereof. The chemical derivative may be carboxymethylpachymaran, hydroxymethyl pachymaran, or hydroxypropyl pachymaran. Alternatively, the beta-1,3-glucan polysaccharide polymer may be a curdlan polymer or a carboxymethyl curdlan polymer.

The beta-1,4-glucan polymer may be a cellulose polymer, such as a carboxymethyl cellulose polymer, chitin or a chitin derivative.

In yet another aspect, there is provided a kit for releasing an active agent from an ionically cross-linked polysaccharide-based polymer hydrogel that encapsulates the active agent. The kit includes: the ionically cross-linked polysaccharide-based polymer hydrogel that encapsulates the active agent; and a chelating agent adapted to chelate at least a portion of the ions that ionically cross-link the polysaccharide-based polymer hydrogel.

In a further aspect, there is provided a method of preparing a polysaccharide-based polymer hydrogel for delivery of an active agent. The method includes: providing a polysaccharide polymer; providing a solution that includes the active agent; dispersing or dissolving the polysaccharide polymer in the solution to form a polymer gel solution; and cross-linking the polysaccharide polymer in the polymer gel solution with a cross-linker to form the polysaccharide-based polymer hydrogel which encapsulates the active agent.

The cross-linker may be an ion and the polysaccharide-based polymer hydrogel may be an ionically cross-linked hydrogel, and the method may accordingly include contacting the polymer gel solution with the ion to crosslink the polysaccharide polymer and form the ionically cross-linked polysaccharide-based polymer hydrogel. The ion may be a metal ion, such as a calcium ion, iron ion, aluminum ion, nickel ion, cobalt ion, or copper ion.

The active agent may be water. The active agent may additionally include a crop protecting agent, such as: a salt, ion, mineral, fertilizer, nematicide, pesticide, herbicide, insecticide, essential nutrient, non-essential nutrient, nucleic acid, fungicide, or any combination thereof. In specific examples, the active agent additionally comprises a plant fertilizer.

In specific examples, the crop protection agent may be a nucleic acid. The nucleic acid may be dispersed in deionized water prior to being added to the polymer solution.

The method may further include drying the hydrogel.

In yet another aspect, there is provided a method of delivering an active agent to a plant. The method includes administering to the plant a hydrogel as described above.

The cross-linker may be a chemical cross-linker and the polysaccharide-based polymer hydrogel may be a chemically cross-linked hydrogel. The method may accordingly include allowing the active agent to diffuse out of the hydrogel.

The hydrogel may further include an excipient, where the active agent is a crop protecting agent, and the method includes osmotic pressure driven release of the active agent from the hydrogel.

In still another aspect, there is provided a method of delivering an active agent to a plant. The method includes administering to the plant an ionically cross-linked hydrogel as described above, and the method further includes administering a chelator to chelate at least a portion of the ions that ionically cross-link the hydrogel and trigger release of the active agent from the hydrogel.

The ions may be are calcium ions, iron ions, aluminum ions, nickel ions, cobalt ions, copper ions or any combination thereof.

The chelator may be sodium citrate, ethylene-diaminetetraacetic acid (EDTA) or a phosphonate.

The ions may be calcium ions and the chelator may be sodium citrate.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 includes photographs of liquid crystalline hydrogel with different amounts of Curdlan and DNA. The amorphous phase of Curdlan appears as white whereas the crystalline phase is opaque. Incorporation of DNA alters the distribution of the amorphous and crystalline phases.

FIG. 2 is a graph illustrating the swelling capacity of Curdlan hydrogel. The figure shows swelling and drying behavior of a piece of Curdlan hydrogel over 100 hours, as measured by weighing the mass of the hydrogel after removal of residual water.

FIG. 3 illustrates the change in mass of CMC hydrogel with repeated swelling and drying cycles over 74 days.

FIG. 4 illustrates the change in mass of CMC hydrogel when place in soil. CMC hydrogels were placed in the soil and tested for stability with varying water administration frequencies.

FIG. 5 depicts the DNA Distribution within a Curdlan hydrogel. Pure Curdlan shows very little absorbance at 260 nm and the overall absorbance shifts higher with greater incorporation of DNA. At the highest DNA loading, two ‘rings’ of DNA are evident in the structure.

FIG. 6 includes photographs of Curdlan hydrogel millispheres and illustrates the effect of DNA and calcium chloride concentration on the millispheres. With decreasing DNA concentration, the resilience of the hydrogel spheres decreases until no visible sphere forms. Reduction of calcium chloride concentration reduces the fiber density, causing the spheres to exhibit more swelling.

FIG. 7 shows optical microscopy micrographs of Curdlan hydrogel millispheres formed with varying concentrations of DNA and calcium chloride. Optical microscopy at 10× magnification reveals the presence of two interfaces within the millispheres as was visible in natural light but this phenomena only occurs with DNA loading greater than 50%.

FIG. 8 shows a transmission electron microscopy micrographs of Curdlan hydrogels. At high concentration, Curdlan forms microspheres encapsulating crystallized DNA. At low concentrations, Curdlan forms nanofibrous networks that are capable of incorporating DNA as globular spherical particles. Further increases in DNA yield nanoparticles and longer rigid rod-like structures. For samples with 0.5 and 2.5 mg/mL DNA, TEM micrographs are taken from the supernatant. Scale bars are 500 nm.

FIG. 9 is a graph illustrating release of DNA from Curdlan hydrogel using sodium citrate. No release occurs in deionized water, whereas the graph illustrates a considerably higher release rate when the hydrogel is placed in 1% sodium citrate. Inset plot shows a single hydrogel sample moved from water to sodium citrate to demonstrate triggered release by the addition of an external triggering agent.

FIG. 10 is a graph illustrating release of fertilizer from CMC hydrogel. CMC hydrogel was placed in deionized water and release of fertilizer was observed by diffusion.

FIG. 11 is a graph illustrating release of hydrophobic and hydrophilic molecules. CMC hydrogel was used to encapsulate Fast green FCF and methylene blue and it was observed that both dyes had a similar rate of release.

FIG. 12 is a graph illustrating wheat growth using CMC fertilizer hydrogel. CMC hydrogel encapsulating fertilizer was implanted with wheat seeds. Data presented is average height normalized against maximum height from control experiments where wheat seeds were grown without the CMC hydrogel. Error bars are standard error of mean (n=6 for CMC hydrogel, n=10 for control).

FIG. 13 is a graph illustrating canola growth using CMC fertilizer hydrogel. CMC hydrogel encapsulating fertilizer was implanted with canola seeds. Data presented is average height normalized against maximum height from control experiments where canola seeds were grown without the CMC hydrogel. Error bars are standard error of mean (n=6).

FIG. 14 is a graph illustrating wheat growth using CMC fertilizer hydrogel with crosslinking performed at 40° C. Data presented is average height normalized against the maximum height of wheat seeds which were grown with fertilizer but without hydrogel. Error bars are standard error of mean (n=6).

FIG. 15 is a graph illustrating wheat growth using ionically cross-linked CMC fertilizer hydrogel dried at 80° C. Data presented is average height normalized against the maximum height of wheat seeds which were grown with fertilizer but without hydrogel. Error bars are standard error of mean (n=6).

FIG. 16 is a graph illustrating wheat growth using chemically cross-linked CMC fertilizer hydrogel dried at 80° C. Data presented is average height normalized against the maximum height of wheat seeds which were grown with fertilizer but without hydrogel. Error bars are standard error of mean (n=6).

FIG. 17 is a graph illustrating wheat growth using ionically cross-linked CMC fertilizer hydrogel with plants given water twice weekly as opposed to daily (2/7 total water volume). Data presented is average height normalized against the maximum height of wheat seeds which were grown with fertilizer but without hydrogel.

FIG. 18 is a graph illustrating wheat growth using chemically cross-linked CMC fertilizer hydrogel with plants given water twice weekly as opposed to daily (2/7 total water volume). Data presented is average height normalized against the maximum height of wheat seeds which were grown with fertilizer but without hydrogel. Error bars are standard error of mean (n=6).

### DETAILED DESCRIPTION

The present disclosure provides a polysaccharide-based hydrogel polymer having an encapsulated active agent for delivery to a plant, animal or human.

The polysaccharide-based hydrogel may be, for example, a peptidoglycan polymer hydrogel, an alpha-glucan hydrogel or a beta-glucan hydrogel, that can be loaded with, or encapsulate, one or more active agents. The polysaccharide-based hydrogel polymer may be prepared, for example, through addition of aqueous solutions of polysaccharide polymer to an aqueous salt solution to produce an ionically cross-linked hydrogel.

In another example, the polysaccharide-based hydrogel polymer may be prepared through addition of aqueous solutions of polysaccharide polymer with a chemical cross-linker, such as glutaraldehyde, to produce a chemically cross-linked hydrogel. Other multifunctional aldehyde or carboxylic acid molecules may also be used to form chemical crosslinks through the formation of ether or ester bonds with hydroxyl and carboxylic acid groups present on the polysaccharide backbone. Examples of these chemicals include, but are not limited to: glutaraldehyde, succindialdehyde, citric acid, maleic acid and itaconic acid. In addition, multifunctional amines or amides, such as tetramethylolacetylenediurea, may also be used to achieve crosslinking of polysaccharides through the formation of secondary amines or amides. Other compounds which may be used to form chemical crosslinks include multifunctional compounds that have two or more chemical groups that react with hydroxyl or carboxylic acid groups present on the polysaccharide backbone. One example of such a compound is toluene diisocyanate.

The beta-glucan hydrogel may be, for example, a beta-1,3-glucan hydrogel prepared, for example, by re-naturing water insoluble beta-1,3-glucan from solution in the presence of an aqueous metallic salt. The beta-1,3-glucan may be “curdlan” and the beta-1,3-glucan hydrogel may correspondingly be called a “curdlan” hydrogel. In another example, the beta-glucan hydrogel may be a beta-1,4-glucan hydrogel prepared by the ionic cross-linking of a water soluble derivative of beta-1,4-glucan. The beta-1,4-glucan may be “cellulose” and the beta-1,4-glucan hydrogel may correspondingly be called a “cellulose” hydrogel. One example of a beta-1,4-glucan cellulose hydrogel is “carboxymethyl cellulose” hydrogel (CMC hydrogel).

Polysaccharide-based hydrogel polymers may protect the active agent from degradation before the active agent is released. For example: Lipophilic drugs, such as, for example: pesticides, fungicides, insecticides, growth hormones, and draught protection agents; hydrophilic drugs, such as, for example: salts, ions, minerals, essential nutrients and non-essential nutrients; peptide drugs; protein drugs; growth hormones; growth factors; or combinations thereof; may be encapsulated in the polysaccharide-based hydrogel polymer to reduce the rate of their degradation before being released.

Polysaccharide-based hydrogel polymers made using biodegradable polymers and/or monomers may be degraded, metabolized by microbes, or both degraded and metabolized over time. For example, polysaccharide-based hydrogel polymers may be degraded within 2 years following administration to plants. In some examples, the polysaccharide-based hydrogel polymers may be degraded within several weeks following administration to plants. The polysaccharide-based hydrogel polymers may be degraded via hydrolysis of the polymeric bonds.

Polysaccharide-based hydrogel polymers having an encapsulated active agent may reduce the amount of the active agent delivered the soil and increase the amount of the active agent delivered to the plant, in comparison to the amounts of active agent delivered to the soil and delivered to the plant when the active agent is administered without being encapsulated in the polysaccharide-based hydrogel polymer.

Polysaccharide-based hydrogel polymers may actively release the encapsulated agent, for example through a release triggered by the addition of an external triggering agent or through a release triggered by an osmotic pressure driven release mechanism. Alternatively, polysaccharide-based hydrogel polymers may passively release the encapsulated agent, for example through diffusion of the active agent out of the polysaccharide-based hydrogel polymer. Triggered release by the addition of an external triggering agent would be understood to refer to the de-crosslinking of the hydrogel, thereby resulting in non-crosslinked polysaccharide-based polymers, and the corresponding release of the encapsulated agent from the hydrogel. One example of such a triggered release is the de-crosslinking of an ionically crosslinked hydrogel using a chelator that interacts with the ions to prevent the ions from crosslinking the hydrogel. As the crosslinking ions are disassociated from the hydrogel by the chelator, the hydrogel is de-crosslinked and releases the encapsulated agent. A “chelator” may also be referred to as a “chelating agent”.

Polysaccharide-based hydrogel polymers may be used in dry conditions to provide a supply of active agent. For example, hydrogels may act as a reservoir for the supply of water. The hydrogel may be tailored such that various amount of water can be delivered, for example ranging from 0.3 mg water per gram of hydrogel to 3 g of water per gram of hydrogel.

Polysaccharide-based hydrogel polymers may be directly implanted in root beds to allow delivery of water and other active agents to a plant; or may be sprayed on plants to deliver the encapsulated active agent to the leaves of a plant.

Excipients may be included with the polysaccharide-based hydrogel polymers when encapsulating active agents which are sensitive a component used in the production of the polysaccharide-based hydrogel polymers. For example, excipients may be included when the active agent is sensitive to alkaline medium or high ionic content. One example of such an excipient is a surfactant, such as Tween-60, which may be included for active agents that are hydrophobic and not soluble in alkaline medium.

A coating may be applied to the polysaccharide-based hydrogel polymer, if desired.

As used herein, “plant” refers to any type of plant, including but not limited to, trees, flowers, bushes, grasses, vines, ferns, mosses, and the like, including, for example, flowering plants and plants bearing fruit, seeds, legumes, cereals, tubers and the like. The term “plant” includes crops. The term “plant” includes “plant portion”, such as a root, stem or leaf.

As used herein, “crop” refers to a plant species or variety that is grown to be harvested as food, livestock fodder, fuel or for any other economic purpose.

In the context of the present disclosure, a “hydrogel”, also termed a “hydrogel polymer”, “polysaccharide-based hydrogel”, or “polysaccharide-based hydrogel polymer”, is formed from a network of polysaccharide-based polymer chains that are hydrophilic, sometimes found as a colloidal gel in which water is the dispersion medium. These polysaccharide-based polymers may be cross-linked, for example by metallic ions, chemical cross-linkers or hydrogen bonding. Hydrogels or hydrogel polymers include highly absorbent natural or synthetic polymers and may contain over 99 wt % water when fully hydrated. Hydrogels may possess a degree of flexibility due to their water content.

Polysaccharides polymers are polymeric carbohydrate structures formed of repeating units (either mono- or di-saccharides) joined together by glycosidic bonds. In some examples disclosed herein, the polysaccharide polymer is a beta-glucan polymer, for example curdlan or cellulose, such as carboxymethyl cellulose (CMC). Other polysaccharides that could be employed include alpha-glucan. One such alpha-glucan is alpha-1,6-glucan with alpha-1,3 branches, known as dextran. For example, if polyaldehyde dextran is used as the polysaccharide polymer, an amine based cross-linking agent, such as ethylenediamine, could be used to create the hydrogel. Multifunctional azide-based compounds could also be used as cross-linkers for polyandehyde dextran hydrogels by employing click chemistry to crosslink the hydrogel. Other alpha-glucans, including alpha-1,4-; alpha-1,6-glucans such as pullulan and starch, can also form hydrogels using ionic or chemical crosslinkers. When considering beta-glucans, curdlan is a linear 1,3-beta-glucan, other 1,3-gbeta-glucans with 1,6-branches are also expected to form hydrogels because they exhibit similar properties as curdlan. These branched polysaccharides include schizophyllan, lentinan, pachyman, pachymaran, scleroglucan, grifolan and their chemical derivatives such as carboxymethylpachymaran, hydroxymethyl pachymaran, hydroxypropyl pachymaran and carboxymethyl curdlan. Other 1,4-vbeta-glucans are also expected to form hydrogels using ionic or chemical crosslinkers due to their similarities with carboxymethyl cellulose. Some examples of such 1,4-beta-glucans include chitin and its derivatives. Additionally, it is expected that peptidoglycans can also form hydrogels with enhanced properties where the peptides will assist in the ionic co-ordination based on their charge. The peptide units can also provide specific functionalities, such as creation of hydrophobic pockets for encapsulation of active agents.

Polysaccharide polymers which may be used to form the hydrogels according to the present disclosure may have a molecular weight from 10 kDa to 5,000 kDa. In particular formulations used to prepare the hydrogels, the polysaccharide is at a concentration of at least 1 mg/mL, and preferably at 70 mg/mL. The polysaccharides have a molecular weight of at least 10 kDa, and preferably 250 kDa (this is the molecular weight of CMC used in the examples). If the hydrogel is an ionically crosslinked hydrogel, the polysaccharide needs to be soluble in an aqueous solution. The hydrogel does not need to be soluble in an aqueous solution if alternative hydrogels are being formed. If the polysaccharide polymer is not water soluble, derivatives may be obtained to increase the solubility and allow the polysaccharide polymer derivative to be ionically cross-linked. In particular embodiments used to prepare ionically cross-linked hydrogels, the concentration of ions in the cross-linking solution is at least 0.01 wt %, and preferably 10 wt %.

As used herein, “curdlan” (or beta-1,3-glucan) refers to a high molecular weight polymer of glucose comprising beta-(1,3)-linked glucose residues capable of forming an elastic hydrogel upon heating in an aqueous suspension. It may be produced by Agrobacterium biobar, a non pathogenic bacteria. Alternatively, curdlan may be produced by Alcaligenes faecalis. High molecular weight curdlan can range anywhere from 1×10⁵ Da to 30×10⁵ Da. Curdlan having a molecular weight at the upper end of this range may offer additional benefits to the resulting hydrogel. (Dobashi, 2004; Nobe, 2005).

As used herein, the term “carboxymethyl cellulose” (or CMC) refers to a high molecular weight cellulose (that is, a beta-1,4-glucan) polymer derivative comprising carboxymethyl groups (—CH₂—COOH) bound to some of the hydroxyl groups of the glucopyranose monomers that make up the cellulose backbone. High molecular weight CMC can range anywhere from 90 kDa to 700 kDa. The degree of substitution of CMC may range from 0.1 to 2.8, with the preferred degree of substitution being 0.7 carboxymethyl groups per anhydroglucose unit. CMC polymers which may be used to prepare hydrogels according to the present disclosure have been previously discussed in U.S. Pat. No. 4,618,491 to Kanematu, T. and Yamaguchi, Y.

As used herein, the term “active agent” includes any agent that has a desired effect on plants, animals or humans. This includes, but is not limited to, for example: small molecules, such as pharmacologically active compounds for example hydrophobic or hydrophilic drugs; immunostimulators; anti-cancer molecules; vaccines; biopolymers, such as peptides, proteins, polynucleic acids, for example: peptide drugs, protein drugs, growth hormones, growth factors; crop protecting agents such as, for example: salts, ions, minerals, fertilizers, nematicides, pesticides, herbicide, insecticides, essential nutrients, non-essential nutrients, nucleic acid, or fungicides; or draught protecting agents, such as water. It would be understood that the properties of the active agent to be delivered may impact which polysaccharide-based hydrogel polymer is used for the delivery. For example, if water was to be delivered to a plant, a more hydrophobic hydrogel may be selected since more hydrophobic hydrogels may contain as much as 99 wt % water.

Nutrients may include macronutrients or micronutrients. As used herein, “macronutrients” (e.g. nitrogen, phosphorus, potassium, calcium, magnesium and sulfur) are plant nutrients required in the largest amount in plants, whereas “micronutrients” (e.g. iron, copper, manganese, zinc, boron, molybdenum and chlorine) are required in relatively smaller amounts. Additional mineral nutrient elements which are beneficial to plants but not necessarily essential include sodium, cobalt, vanadium, nickel, selenium, aluminum and silicon. The nutrient elements differ in the form they are absorbed by the plant, by their functions in the plant, by their mobility in the plant and by the plant deficiency or toxicity symptoms characteristic of the nutrient.

As used herein, “crop protection agent” refers to an agent that directly or indirectly promotes the heath or growth of a plant, including but not limited to, fertilizers, fungicides, pesticides, herbicides, nematicides, insecticides, nematicides and nucleic acids. A hydrogel according to the present disclosure may allow the release of active agent encapsulated by the hydrogel, for example by utilizing a triggered release effect dependent on the ionic nature of the physical structure of the hydrogel, or by utilizing an osmotic pressure driven release mechanism.

A polysaccharide-based hydrogel polymer encapsulating an active agent may be formed, for example, through the addition of the active agent to be encapsulated to a mixture of curdlan (beta-1,3-glucan) in an alkaline solution. In such an alkaline solution, curdlan is denatured. The mixture may then, for example, be re-natured upon the addition of aqueous metal salts which ionically cross-link helical domains of the curdlan and form a polysaccharide-based hydrogel polymer which encapsulates the active agent. The encapsulated active agent may be hydrophilic or hydrophobic. The hydrogel may be formed in a variety of shapes, sizes, and forms. Adding a chelating agent to the ionically cross-linked hydrogel triggers the release of the active agent from the curdlan hydrogel since the chelating agent binds to the metal salts and denatures the curdlan hydrogel. A curdlan hydrogel may alternatively be formed using chemical cross-linking molecules that react with the hydroxyl groups on the polysaccharide polymer backbone. The active agent can be loaded into chemically crosslinked curdlan hydrogels after the crosslinking reaction using, for example, a process called reverse loading discussed below.

These curdlan hydrogels possess a very high swelling capacity, in some examples reaching a mass of up to 20 times its dry weight upon hydration, making them highly applicable to retaining and releasing water in soil. Curdlan hydrogels are capable of delivering of a number of other active agents applicable to crop protection, including, for example: pesticides, herbicides, fungicides, and fertilizers for crop protection and growth, and nucleic acids for transgenic applications. The use of different methods of addition and concentrations of curdlan, active agent, and salt allow for the formation of a variety of structures from, for example, nanofibrous networks to microparticles and macroscopic cylindrical hydrogels. Examples of different hydrogels formed with various concentrations of curdlan, active agent and/or salt are discussed in Examples 1, 2, 8 and 9, as well as the corresponding figures related to the Examples.

Sodium carboxymethyl cellulose (CMC) hydrogel polymers are other examples of polysaccharide-based hydrogel polymers which may be used to encapsulate an active agent. For example, CMC hydrogels may be used to encapsulate water for delivery to plants. CMC hydrogels may be biodegraded after the CMC hydrogel has been implanted in the desired application.

CMC hydrogels may be produced, for example, from an aqueous solution of CMC and the active agent, which are then added to an aqueous salt solution, thereby producing an ionically cross-linked CMC hydrogel which encapsulates the active agent. The encapsulated active agent may be hydrophilic or hydrophobic.

In an alternative example, CMC hydrogels maybe made using chemical cross-linking. Chemical cross-linked CMC hydrogel may be made, for example, by using a solution of glutaraldehyde, water and hydrochloric acid, instead of iron and calcium salts. This results in a CMC hydrogel with a much larger pore size and capacity for swelling due to water absorption. The different nature of the crosslinks also eliminates the chelating effect of active agent loading since the active agent can be loaded after the crosslinking reaction using, for example, a process called reverse loading.

Reverse loading is done by dissolving the desired active agent, such as 20/20/20 fertilizer in water, and allowing the dissolved active agent to diffuse into the hydrogel. Dehydrating the loaded hydrogel through drying retightens the pores of the hydrogel and allows controlled release of the loaded active agent.

Hydrogels that are chemically cross-linked, for example using glutaraldehyde, have a much higher capacity for loading than ionic cross-linked hydrogels. Release testing of the ionic cross-linked hydrogels showed that the ionically crosslinked hydrogels failed to prevent a burst release of fertilizer upon being first placed into soil once fertilizer loading exceeded 40 wt %. On the other hand, chemically cross-linked hydrogels were able to accommodate fertilizer loadings in excess of 90 wt % fertilizer, while still maintaining controlled release.

CMC hydrogels, or other hydrogels formed from polysaccharide polymers having carboxyl groups on the polymer backbone, may be formed using cross-linking molecules that react with the carboxyl groups. The increased reaction rate and higher crosslinking density may provide advantages over hydrogels formed using cross-linking molecules that react with hydroxyl groups on the polymer backbone. However, cross-linkers that react with hydroxyl groups on the polymer backbone, rather than the negatively charged carboxyl groups, allow polysaccharides such as curdlan or cellulose to be used in the place of carboxymethyl cellulose. As with CMC and curdlan, cellulose hydrogels may be formed by dissolving cellulose in an aqueous NaOH solution, thereby denaturing the polysaccharide polymers, and re-naturing the polymers through the addition of metal cations to form an ionically cross-linked hydrogel.

CMC and cellulose hydrogels may be used to deliver a number of active agents applicable to crop protection, including, for example: water, pesticides, herbicides, fungicides, and fertilizers for crop protection and growth, and nucleic acids for transgenic applications. The use of different methods of addition and concentrations of CMC or cellulose, active agent, chemical crosslinker and/or salt allow for the formation of a variety of structures from, for example, nanofibrous networks to microparticles and macroscopic cylindrical hydrogels. Examples of different hydrogels formed with various concentrations of CMC, active agent, chemical crosslinker and/or salt are discussed in Examples 3, 7, 10, 12 and 13 as well as the corresponding figures related to the Examples.

Based on the results of curdlan hydrogels, it is believed that adding a chelating agent to the ionically cross-linked CMC hydrogel may trigger the release of the active agent from the CMC hydrogel since the chelating agent would bind to the metal salts which ionically crosslink the CMC hydrogel and thereby denature the CMC hydrogel.

Drying hydrogels, such as CMC or cellulose hydrogels, may aid in preventing burst release of encapsulated active agent. Drying of CMC or cellulose hydrogels may be done by placing the hydrogels—after crosslinking and active agent loading—into an oven at about 80° C. for an extended period of time. The drying of the hydrogels induces crystallization of the polysaccharide polymers which reduces the material's pore size and increases its resistance to chemical degradation from water. Drying for 48 h provides the maximum amount of crystallization of the CMC or cellulose hydrogel. Curdlan hydrogels may also be dried and it is expected that drying curdlan hydrogels would affect the release rate of the active agent.

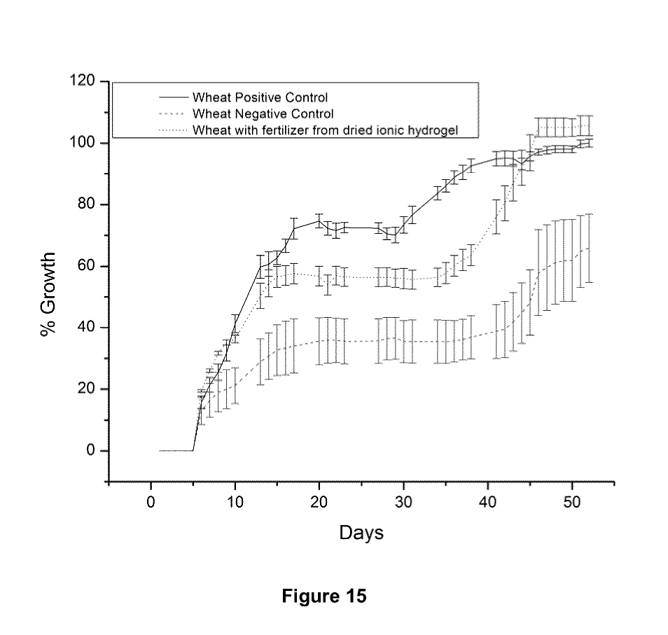

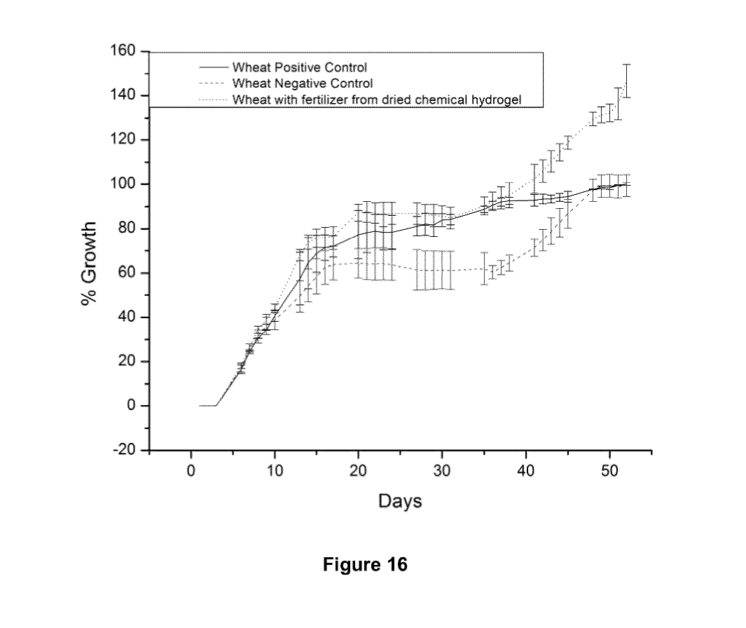

Dried hydrogels can release encapsulated active agent over a much longer timeframe and in a more controlled manner than undried hydrogels. Using dried hydrogels encapsulated with fertilizer, wheat plants have been able to show growth surpassing that of the positive controls, even when the hydrogels included fertilizer doses as low as 21% of the dose applied to the positive controls. These results are illustrated in FIGS. 15 and 16.

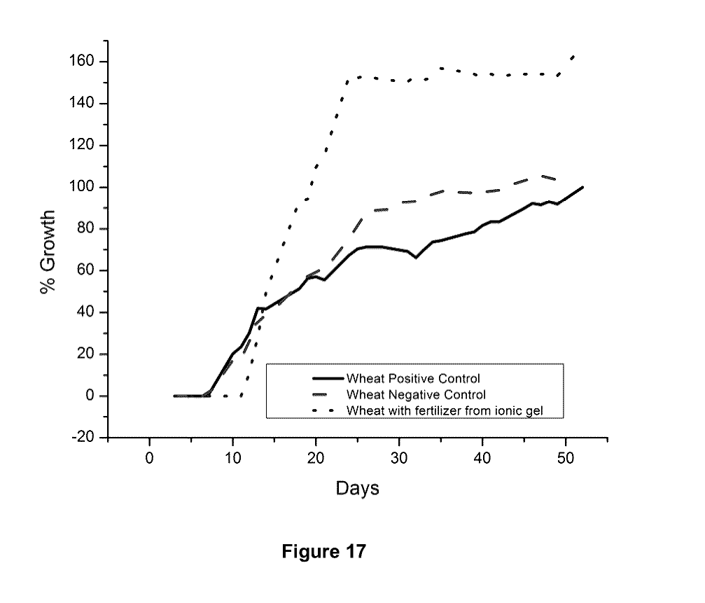

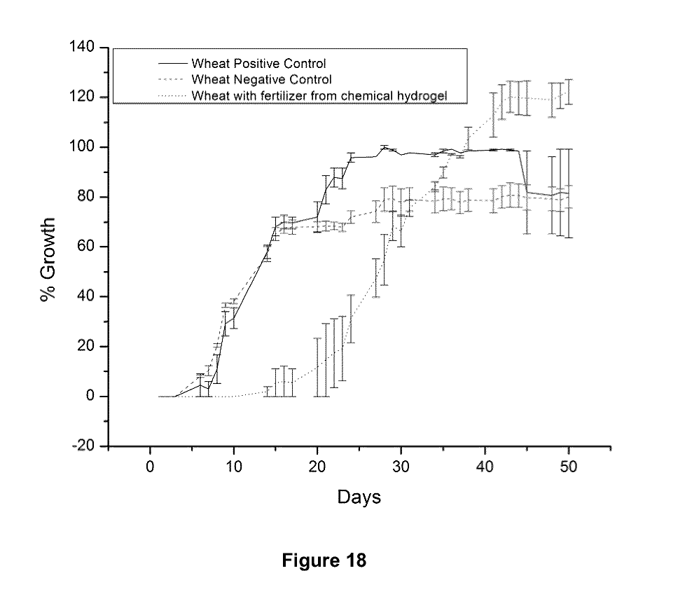

Polysaccharide hydrogels possess the ability to absorb and rerelease water to allow sustained release of water, such as water absorbed during periods of rainfall. The hydrogel's ability to swell and absorb water is dependent on the cross-linking density of the hydrogel. This ability may be used to help maintain the growth of plants, such as crops or turf grass. The CMC hydrogels are able to sustain the health of plants subjected to drought conditions without causing increased soil salinity or changes in soil pH associated with most common soil amendment products. Experiments using CMC hydrogels in drought conditions showed that the CMC hydrogels were able to vastly increase the health and growth of plants relative to controls not treated with CMC hydrogels—not only in terms of height but also plant vigour and colour. The results of this experiment are illustrated in FIGS. 17 and 18.

After polysaccharide-based polymers form a gel structure, for example on dissolution in water, polysaccharide-based hydrogel polymers according to the present disclosure may be formed by cross-linking the gel structure, such as by ionic cross-linking, chemical cross-linking, or heat-based cross-linking.

Ionic cross-linking of hydrogel polymers utilizes the presence of chemical groups on the polysaccharide polymer backbone to react with added ions to cross-link the polymers. When the hydrogel polymer includes chemical groups that are hydroxyl groups or carboxylate groups (for example curdlan, cellulose or carboxymethyl cellulose), the cross-linking ion may be a cation. When the hydrogel polymer includes positively charged chemical groups, the cross-linking ion may be an anion.

Polysaccharide polymer chains have a tendency to form hydrogen bonds, in some cases resulting in helical domains with one or three polymer chains in aqueous solution, dependent on hydrogen bonding between hydroxyl groups. The dissolution of the polysaccharide polymer, such as curdlan or cellulose, in dimethyl sulfoxide or an aqueous alkaline solution, such as sodium hydroxide, inhibits hydrogen bonding, resulting in a random coil state. For example, the random coil state may be induced with sodium hydroxide at a concentration above 0.2M.

In formulations where the gel structure is dissolved in an aqueous solution and the polymer includes hydroxyl and/or carboxylate groups, the random coils of the denatured polysaccharide polymers may be re-natured to form helical domains through the addition of metal cations which form bonds between deprotonated hydroxyls and/or carboxylate groups in the polysaccharide polymer chains to cross-link those domains.

The metal cations may be, for example, ions of calcium, cobalt, aluminum, nickel, or iron. For example, iron cations may be obtained from iron(II) chloride, or iron (III) chloride. Aluminum ions may, for example, be obtained from aluminum chloride. In some examples, the cation is a divalent cation, such as calcium cations. Positive ions, such as ions of calcium, cobalt, aluminum, nickel or iron, can also be used for ionic cross-linking of CMC by cross-linking hydroxyls on the CMC polymer backbone. Ionic cross-linking may take place at room temperature.

Heating may alternatively be used to cross-link polysaccharide-based hydrogels, such as curdlan, cellulose or CMC, without the addition of positive metal ions. Hence, cross-linking can also be conducted at higher temperatures. For example, CMC hydrogels may be formed by heating CMC at 40° C. in water.

Alternatively, polysaccharide-based hydrogels maybe made using chemical cross-linking. Chemical cross-linked hydrogels may be made by using, for example, a solution of glutaraldehyde, water and hydrochloric acid, instead of iron and calcium salts. This results in a hydrogel with a much larger pore size and capacity for swelling due to water absorption. The different nature of the crosslinks also eliminates the chelating effect of active agent loading since the active agent can be loaded after the crosslinking reaction using, for example, a process called reverse loading. Dehydrating the loaded hydrogel through drying retightens the pores of the hydrogel and allows controlled release of the loaded active agent. Hydrogels that are chemically cross-linked, for example using glutaraldehyde, have a much higher capacity for loading than ionic cross-linked hydrogels.

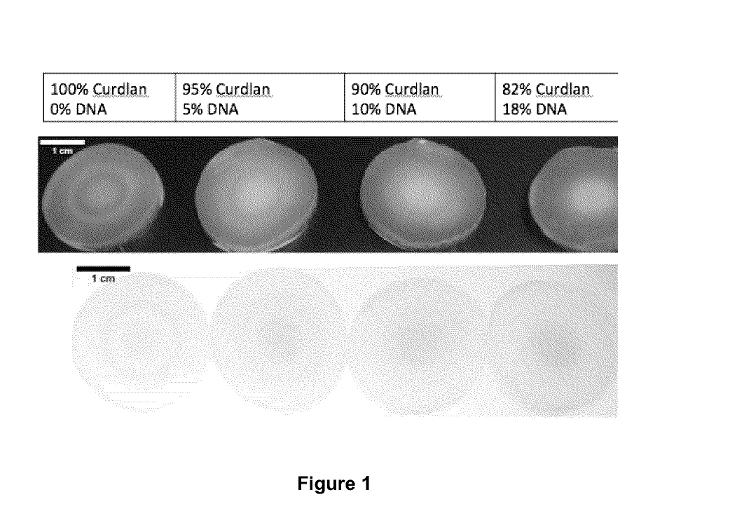

Cross-linking may be used to create physical hydrogels in a wide variety of shapes and forms. FIG. 1 shows ionically cross-linked curdlan with differing levels of encapsulated active agent, where the active agent is DNA ranging from 0% to 18%, as discussed in greater detail in Examples 1 and 2. Curdlan liquid crystalline hydrogel with 0% active agent shows the presence of visible concentric rings as formed from ionic cross-linking. The presence of concentric rings is due to the diffusion gradient of calcium, causing alternating layers of amorphous (white rings) and crystalline curdlan (dark rings). The addition of the active agent causes a unique distribution of the active agent into the curdlan hydrogel. The distribution may be different depending on the molecular weight and crystallinity of the molecule being incorporated.

The active agent may be actively released from the polysaccharide-based hydrogel polymer over time using, for example, an osmotic pressure driven release mechanism. The release of the active agent over time may reduce the need for repeated applications of the active agent when compared to active agents which are administered without the polysaccharide-based hydrogel polymer.

Polysaccharide-based hydrogel polymer which use an osmotic pressure driven release mechanism may additionally include excipients encapsulated in the polysaccharide-based hydrogel polymer to increase the rate of release of the active agent from the polysaccharide-based hydrogel polymer. Excipients used for osmotic pressure driven release, also referred to as bulking agents or osmotagens, increase the rate of release of the active agent by generating osmotic pressure within the polysaccharide-based hydrogel polymer. Sucrose is one example of an excipient which may be used to generate osmotic pressure.

Without wishing to be bound by theory, it is believed that water is osmotically recruited by the excipients which are encapsulated in the polysaccharide-based hydrogel polymer. The active agents which are encapsulated in the polysaccharide-based hydrogel polymer are dissolved by the water diffusing into the polysaccharide-based hydrogel polymer, thereby forming aqueous microcapsules inside the matrix of the polysaccharide-based hydrogel polymer.

Since the polysaccharide-based hydrogel polymer which surrounds the aqueous microcapsule is elastic, water which is recruited by the excipients causes swelling of the polymer. Although the polymer may initially resist the swelling by the polymer elastic strain, the eventual swelling of the microcapsules generates cracks in the polysaccharide-based hydrogel polymer due to bond breakage in the surrounding polysaccharide-based hydrogel polymer. This breakage of the polysaccharide-based hydrogel polymer is at a pressure which can be determined by the concentration of excipient encapsulated by the polysaccharide-based hydrogel polymer.

This swelling of the microcapsules, and the resulting cracking of the polysaccharide-based hydrogel polymer, proceeds in a layer-by-layer fashion through the device, resulting in the release of the active agent. The rate of release of the active agent may be effected by adjusting the osmotic activity inside the polysaccharide-based hydrogel polymer. The osmotic activity may be adjusted by, for example: changing the excipient, changing the amount of excipient, or both. For example, the rate of release can be adjusted to at 0.01 mg of active agent per day per gram of the polysaccharide-based hydrogel polymer for a duration of 1 months to several months.

Polysaccharide-based hydrogel polymers which use an osmotic pressure driven release mechanism may enable active agents to be delivered to plants for the duration of a growing season (for agricultural crops) from a single application of the polysaccharide-based hydrogel polymer. This could reduce the manpower needed for repeated administration, use less of the active agent, or both.

With osmotic pressure driven release mechanisms, polysaccharide-based hydrogel polymers administered to the soil around plants absorb water from the environment into the polysaccharide-based hydrogel polymer by capillary force. The movement of water towards the polysaccharide-based hydrogel polymer creates an osmo-chemo-taxi path for the plant root to navigate towards the polysaccharide-based hydrogel polymer. As the plant roots grows around the polysaccharide-based hydrogel polymer, the plant root hairs can physical grow and attach themselves onto the polysaccharide-based hydrogel polymer. The polysaccharide-based hydrogel polymer may act as a depot to release the active agent encapsulated in the polysaccharide-based hydrogel polymer to the roots.

Hydrogels according to the present disclosure have a physical structure which may be used to encapsulate active agents. The curdlan triple helices of beta-1,3-glucan hydrogel, formed through ionic cross-linking using aqueous calcium chloride, is one example of a polysaccharide-based hydrogel polymer which has a physical structure capable of encapsulating active agents. Other examples of polysaccharide-based hydrogel polymers which have physical structures capable of encapsulating active agents include ionically cross-linked curdlan, CMC or cellulose hydrogel polymers formed using, for example, iron or calcium ions. Varying the concentration of the ions varies the mechanical properties of the resulting CMC, cellulose or curdlan hydrogels and hence varies the delivery characteristics.

Yet other examples of polysaccharide-based hydrogel polymers which have physical structures capable of encapsulating active agents include a curdlan, CMC or cellulose hydrogel crosslinked with a chemical crosslinker, such as glutaraldehyde. Varying the amount of chemical crosslinker varies the mechanical properties of the resulting curdlan, CMC or cellulose hydrogels and hence varies the delivery characteristics.

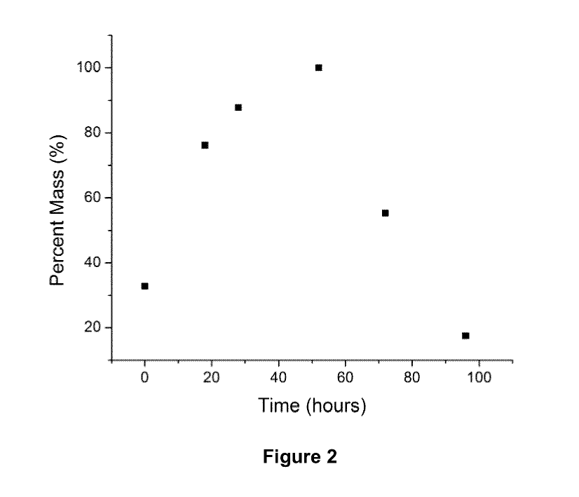

Some molecular characteristics of the active agent (for example, high alkaline solubility and low water solubility) may result in increased encapsulation efficiency. However, other molecules could be encapsulated as well, including small molecules. For application in water retention, it may be desirable for polysaccharide-based hydrogel polymers to have high water swelling capability and consistent repeatability. FIG. 2 shows the progression in the weight of a beta-1,3-glucan hydrogel during a swelling and dehydration cycle after being initially dried. The hydrogel consists of up to ninety percent aqueous medium, (for example, water) that can be removed through simple air-drying. Re-hydration in water causes the hydrogel to re-swell to its original mass. The ability to hold large volumes of water allows the hydrogel to water to a plant. The amount of water retention which is desirable depends on the application. For example, in dry areas where delivery of water is one of the goals, more water retention is desired. On the other hand, in wet areas, less water retention is desirable so as to cause minimal disturbance to soil levels.

In the case of the loading of crop protection agents into the hydrogel, a homogeneous distribution may be observed dependent on the payload's solubility in the initial aqueous solution. Water-soluble fertilizers, such as nitrate and phosphorus compounds, may be loaded homogeneously into the hydrogel. Insoluble pesticides, such as atrazine, may form precipitates within the hydrogel, providing a secondary diffusion-based barrier to release.

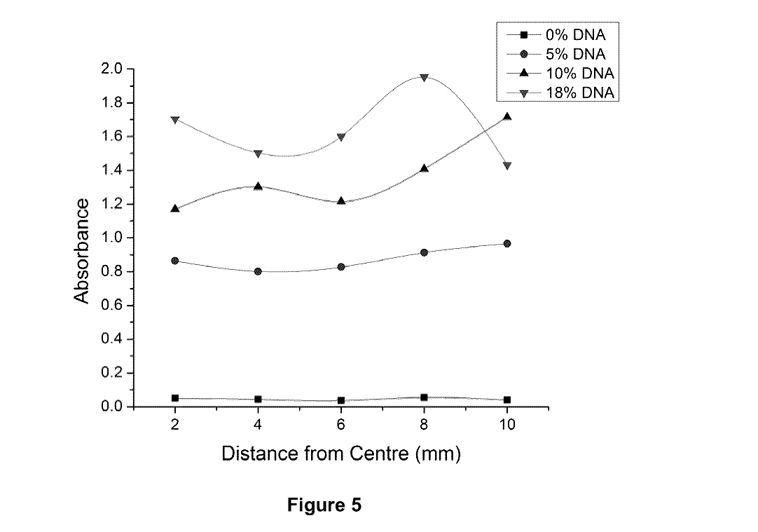

DNA may be encapsulated by polysaccharide-based hydrogel polymers, though it would be understood that DNA is only one example of an active agent which may be encapsulated. DNA is an illustrative example that may be applicable, for example, in the creation of transgenic plants. The distribution of nucleic acids within the hydrogel is affected by the ability of DNA to gel in the presence of metal salts. FIG. 1 shows curdlan liquid crystalline hydrogels with increasing weight percentages of DNA. With the addition of DNA, the hydrogels form amorphous and crystalline phases. With DNA chains which have a molecular weight far exceeding curdlan, the DNA chains in the amorphous state cause a visible increase in the density of the amorphous center, while the crystalline DNA distributes homogeneously in the curdlan matrix. Investigation of the DNA profile across the hydrogels (FIG. 5) confirms this behavior as yielding a distribution of DNA with two distinct concentrations, near the center and near the outer ring. The concentration towards the outside ring exceeds the inner concentration with higher weight percentages of DNA due to the faster gelation rate of DNA (due to a higher molecular weight) as compared to curdlan. With higher DNA concentrations, distribution into the center of the hydrogel is inhibited.

The encapsulated DNA may be CpG DNA, which has been demonstrated to be immunostimulatory. CpG DNA has been previously used in formulations of vaccines. The crystallinity of polysaccharide-based hydrogel polymers, such as curdlan, and DNA on the outer layer provides a protective layer for the internal amorphous DNA. The amorphous DNA would be more bioactive since it is likely to be in its natural form. This configuration provides a stable formulation until the internal DNA needs to be released. The DNA can be released with the assistance of chelators such sodium citrate. In the case of mammals, this could be achieved by administration of oral or intravenous triggering agent such as sodium citrate following the uptake of a hydrogel-DNA system.

Polysaccharide-based hydrogel polymers, such as beta-1,3-glucan hydrogels, may be produced using methods such as dialysis and nanoprecipitation while maintaining their properties at the various scales. The crystallinity of the polysaccharide polymer, such as curdlan, around the centered amorphous DNA may protect the DNA's activity and reduce degradation from enzymes such as DNase. In addition, a polysaccharide polymer, such as curdlan, may have a therapeutic impact which can supplement immunostimulatory DNA's activity.

Polysaccharide-based hydrogel polymers may be formed into a variety of different physical forms. For example, the present disclosure describes curdlan hydrogels in different physical forms. Depending on the concentration, addition of a curdlan solution in a drop-wise manner to stirring aqueous calcium chloride yields the formation of structures ranging from nanofibrous networks to microparticles and larger spherical millispheres. When a liquid solution is preferred to be sprayed over the soil, the nanofibrous networks may be preferred. When the hydrogels are to be used along with plantation of seeds, millispheres and macroscopic hydrogels may be preferred. In case of intravenous administration, nanofibers are likely to be more compatible, while in the case of inhalation, a powder form may be desired. Although the present disclosure may discuss forming physical forms with a specific polysaccharide-based polymer, it would be understood that other polysaccharide-based polymers could also be used to form those physical forms. Accordingly, discussion of curdlan-DNA hydrogels could be replaced with CMC-DNA hydrogels or cellulose-DNA hydrogels, so long as it is understood that the CMC or the cellulose used to form the hydrogels are dissolvable in an aqueous solvent.

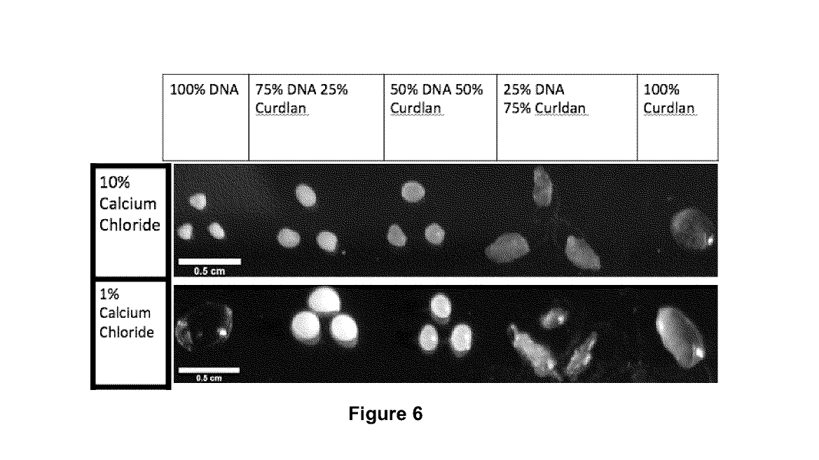

Millispheres are bead like structures having a diameter ranging from 0.1 mm to 10 mm. The simultaneous co-gelation of curdlan with DNA (that is, the formation of a curdlan hydrogel encapsulating DNA) has allowed the formation of millispheres of changing structures dependent on the relative concentrations of curdlan and DNA. This demonstrates the control of the architecture of the structures based on concentration of curdlan and DNA. FIG. 6 demonstrates the evolution of the millisphere structure with changing DNA and calcium chloride concentrations. Millisphere gelation of pure DNA yields solid white hydrogels and increasing concentration of curdlan provides an opaque hydrogel coating, demonstrating that the higher gelation rate of DNA allows it to act as a nucleating center for the millisphere formation. Lowering DNA concentration too much causes a loss of spherical shape as the millisphere is deformed in the stirring process and having no DNA concentration at all yields no such millispheres. Lower concentrations of calcium chloride lower the cross-linking density, causing the particles to swell.

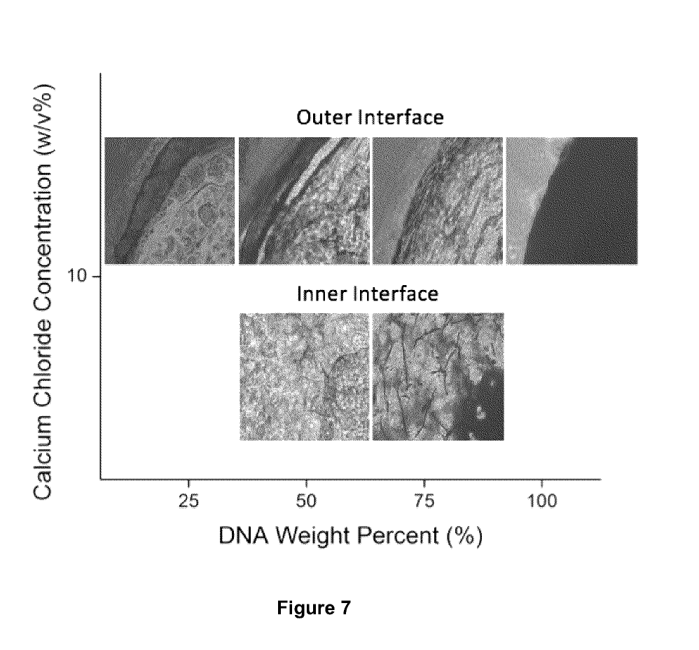

Optical Microscopy reveals the presence of interfaces within the millispheres (FIG. 7). In the case of the 75% DNA system, it was clear that an internal interface divided a central core of DNA, similar in morphology to the pure DNA spheres, and an outer coating of curdlan. A similar interface with more homogeneity was observed in the 50% DNA sample. With lower or higher concentrations of DNA, no internal interface was observed. This indicates the variable distribution of DNA within the millispheres depending on the concentration, which in turn can influence the encapsulation, distribution and release of active agents within the matrix.

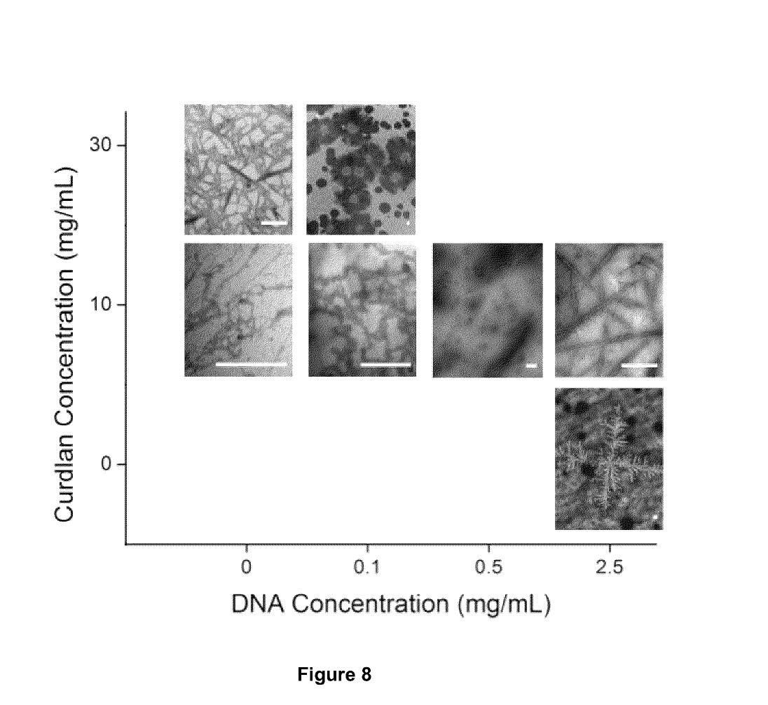

Smaller nano- and microstructures are formed with DNA concentrations an order of magnitude lower. FIG. 8 shows the evolution of the nanostructures obtained from drop-wise addition of mixtures of curdlan and DNA as seen through Transmission Electron Microscopy. At higher concentrations of curdlan, increasing DNA concentration causes a shift from the fibrous structure to the formation of rigid crystalline microparticles with a core-shell structure. At lower concentrations of curdlan, increasing DNA concentration initially causes an increase in fiber density leading to the formation of nanoparticles and rigid rod-like structure bearing a hydrophobic core and hydrophilic shell. However, because the curdlan concentration is lower, visible precipitates are observed in these samples, with the nanostructures being found in the supernatant. At progressively lower curdlan concentrations, DNA crystallites are visible in the samples with no other defining features.

The release of an encapsulated active agent from a polysaccharide-based polymer of the present disclosure may be, for example, triggered by the addition of an external triggering agent. The external triggering agent may be a chelating agent that chelates at least a portion of the ions participating in the cross-linking. Triggered release by the addition of an external triggering agent would be understood to refer to the de-crosslinking of the hydrogel, thereby resulting in non-crosslinked polysaccharide-based polymers, and the corresponding release of the encapsulated agent from the hydrogel.

One example of such a triggered release is the de-crosslinking of an ionically crosslinked hydrogel using a chelator that interacts with the ions to prevent the ions from crosslinking the hydrogel. As the crosslinking ions are disassociated from the hydrogel by the chelator, the hydrogel is de-crosslinked and releases the encapsulated agent. The chelator may be any compound that can chelate the cross-linking ion. In some examples, the chelator may be sodium citrate, ethylenediaminetetraacetic acid (EDTA) or a phosphonate. In some examples, the cross-linking ion may be calcium, iron or copper.

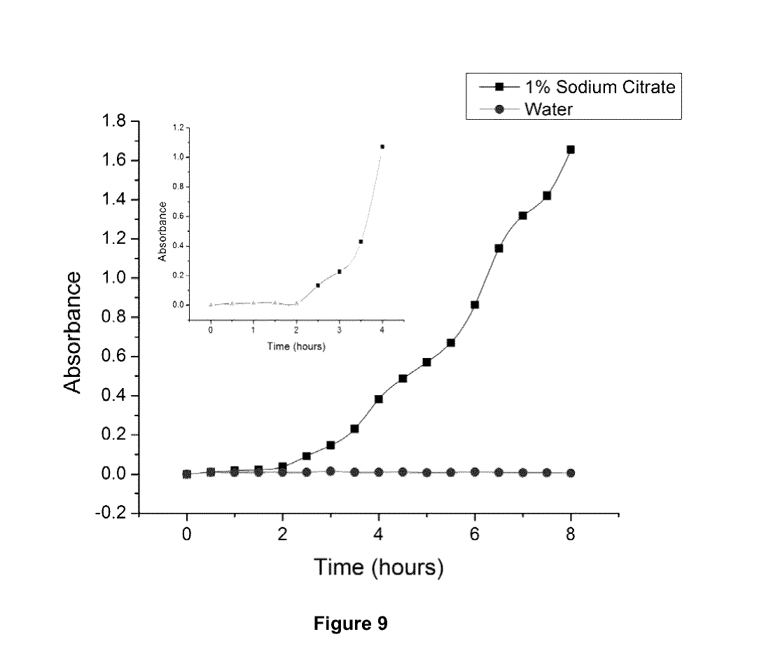

As illustrated in FIG. 9, when molded curdlan hydrogels were placed in water, no release of the DNA payload was observed over 30 hours. With aqueous sodium citrate as the medium, the release profile was drastically different after an initial 2-hour hydration period with the hydrogel almost entirely disintegrating within 8 hours. Using this effect, release may be triggered by moving a hydrogel soaked in water into a sodium citrate medium.

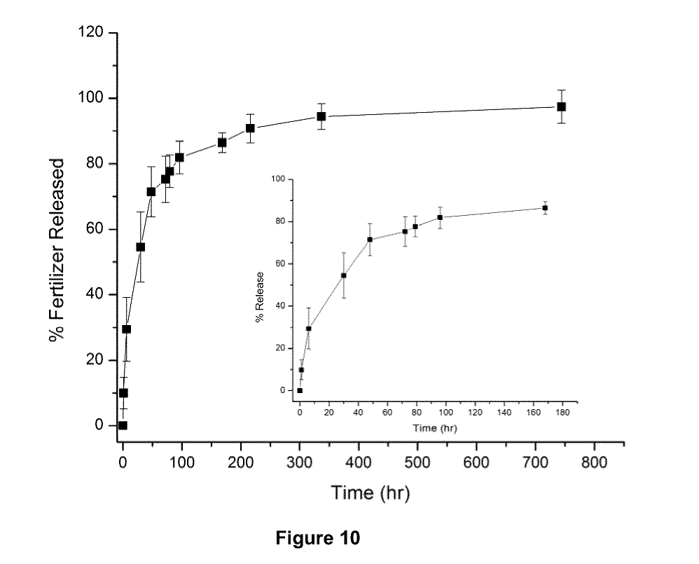

In the case of CMC hydrogels, release of the active agent can be achieved without the presences of a chelating agent. This is illustrated in FIG. 10 using a commercially available fertilizer as the active agent. It was observed that CMC hydrogels could release the fertilizer in deionized water over the period of a month. When applying to soil, the release rate is expected to slow down further because of a lower water content outside the hydrogel.

### EXAMPLES

### Example 1

### Macroscopic Curdlan Liquid Crystalline Hydrogel by Using a Cylindrical Mold

Curdlan obtained from Wako Pure Chemical Industries was dissolved in 0.4M aqueous sodium hydroxide at a concentration of 70 mg/mL. A cylindrical mold was created for the cross-linking process by utilizing a dialysis membrane (Fisherbrand Regenerated Cellulose Dialysis Tubing Flat Width 45 mm and 12,000 to 14,000 Da MWCO) along with two plastic caps of 29.6 mm diameter (Amicon Ultra-15 centrifugal filter unit caps). This apparatus provided a uniform cylindrical shape for synthesizing the hydrogel. Next, 12 mL of Curdlan solution was inserted into the mold by puncturing a hole in one of the caps and later sealing the cap. The dialysis mold with curdlan solution was then placed in 100 mL of 10 wt % aqueous calcium chloride solution for 4 hours. After this duration, the physical cylindrical hydrogel was extracted by cutting the dialysis membrane. A cross-sectional slice of 2 mm thickness was cut from the cylindrical hydrogel to obtain the image in FIG. 1.

### Example 2

### Macroscopic Curdlan-DNA Liquid Crystalline Hydrogel Using Cylindrical Mold

Deoxyribonucleic acid (DNA) sodium salt from salmon testes (Sigma-Aldrich) was dissolved in deionized water at a concentration of 15 mg/mL. Various volumes (3 mL, 5 mL and 7.5 mL) of this DNA solution were added to various volumes (12 mL, 10 mL and 7.5 mL respectively) of 70 mg/mL curdlan solution from example 1 to obtain solutions with 5 wt %, 10 wt % and 18 wt % DNA. Consequently, 12 mL of each of the mixtures was inserted in a separate mold as described in example 1. These samples were placed in 100 mL of 10 wt % aqueous calcium chloride solution for 4 hours. The hydrogels were extracted and a cross-sectional slice of 2 mm thickness was cut to obtain the image in FIG. 1.

### Example 3

### Macroscopic CMC Hydrogels Using a Cylindrical Mold

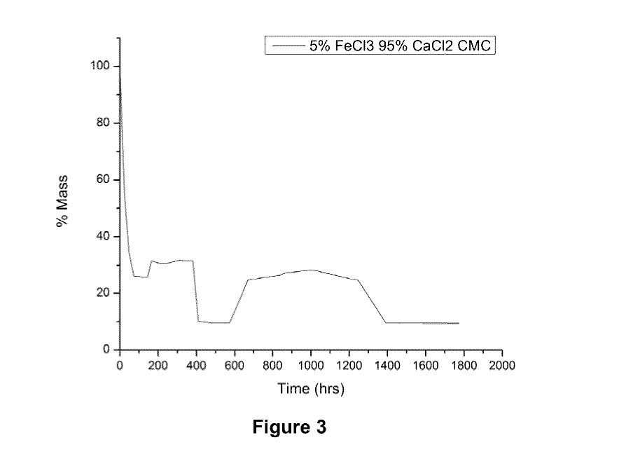

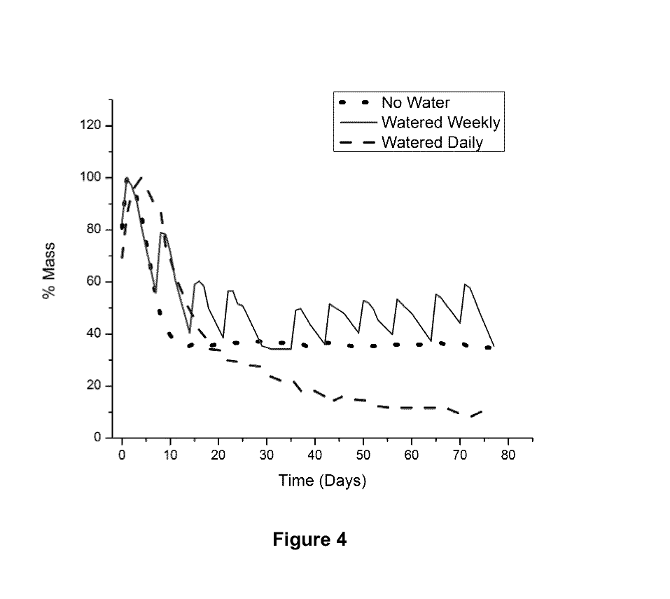

Sodium Carboxymethyl Cellulose (CMC) with a Mw of about 250,000 and 0.7 mol carboxymethyl per mol cellulose (Sigma-Aldrich) was dissolved in deionized water at a concentration of 70 mg/mL. 30 mL of this solution was then transferred to a cylindrical mold as described in Example 1. The dialysis mold with CMC solution was then placed in a solution of varying concentrations of calcium chloride, iron (II) chloride and iron (III) chloride for a period of 72 hours to ensure complete cross-linking. These samples were extracted and transferred to 30 mL deionized water to allow swelling to maximum mass over 72 hours. The mass was recorded and sample left in ambient conditions to dry, until equilibrium was reached. The sample was re-hydrated and the cycle was repeated. The sample synthesized using 0.5 wt % iron (III) chloride and 5 wt % calcium chloride was found to be most stable under swelling and dehydration cycles as highlighted in FIG. 3. In order to extend the swelling and dehydration study in a soil environment for practical applications, the hydrogel was synthesized with 10 wt % 20/20/20 fertilizer encapsulated as highlighted in Example 7, below. This hydrogel was placed in soil and either received no water or was watered daily or weekly. The masses of the hydrogels were measured on a daily basis. Data was normalized against the maximum mass attained for each sample. As illustrated in FIG. 4, the hydrogel remained stable under weekly watered conditions for several swelling cycles.

### Example 4

### Drying and Swelling of Macroscopic Curdlan Hydrogels

Macroscopic cylindrical curdlan hydrogels were synthesized as described in Example 1. Cross-sectional pieces of these hydrogels were obtained with a mass of 1-2 grams. These hydrogels were placed in ambient conditions to dry and in deionized water to swell on a repeated cycle. The mass of the hydrogels was monitored during the drying/swelling cycles using an analytical balance until the mass stopped changing. These results were presented in FIG. 2.

### Example 5

### Distribution of DNA within DNA-Curdlan Macroscopic Hydrogels

DNA-Curdlan hydrogels were synthesized as described in Example 2. A 2 mm thick cross-sectional slice of these hydrogels was obtained which weighed ˜1 grams. This cross-section was further sliced into five 2 mm sections longitudinally starting from the center of the hydrogel and moving outwards. Each of these slices was weighed and dissolved in 5 wt % aqueous sodium citrate solution to obtain a concentration of 10 mg/mL of the hydrogel. These solutions were characterized using UV-Visible spectrophotometry by measuring the absorbance at 260 nm, which is the characteristic absorption peak for DNA. Similar procedure was carried out of a pure curdlan hydrogel for comparison. The absorbance was normalized by subtracting the blank reading of the solvent and then plotted against the distance from the center of the hydrogel. This was seen in FIG. 5.

### Example 6

### Triggered Release of DNA from DNA-Curdlan Macroscopic Hydrogels Using Sodium Citrate

DNA-Curdlan hydrogels were synthesized as described in Example 2. Cross-sectional slices of 2 mm thickness and 1 gram weight were placed in 25 mL of either deionized water or 1 wt % aqueous sodium citrate solution. One of the slices was hydrated for 2 hours in deionized water before transferring to 1 wt % aqueous sodium citrate solution.

Samples were collected from the medium at regular time intervals over a period of 8 hours and the absorbance was measured at 260 nm. The absorbance was normalized by subtracting the absorbance by the solvent and plotted against time of sample collection. These results were shown in FIG. 9. This demonstrated that DNA can be released from the hydrogels by the use of a chelating agent, such as sodium citrate.

### Example 7

### Controlled Release of Agents from CMC Hydrogels

A commercially available 20/20/20 fertilizer was used to demonstrate encapsulation of an active agent in CMC hydrogels. The fertilizer was dissolved in deionized water at a concentration of 140 mg/mL and then added to the CMC solution (70 mg/mL) mentioned in Example 3 such that there was 20 wt % fertilizer with respect to CMC. The dialysis medium contained higher concentration of salt (that is, 1 wt % iron (II) chloride, 1 wt % iron (III) chloride and 10 wt % calcium chloride) than in Example 3 because of the presence of chelators in the fertilizer.

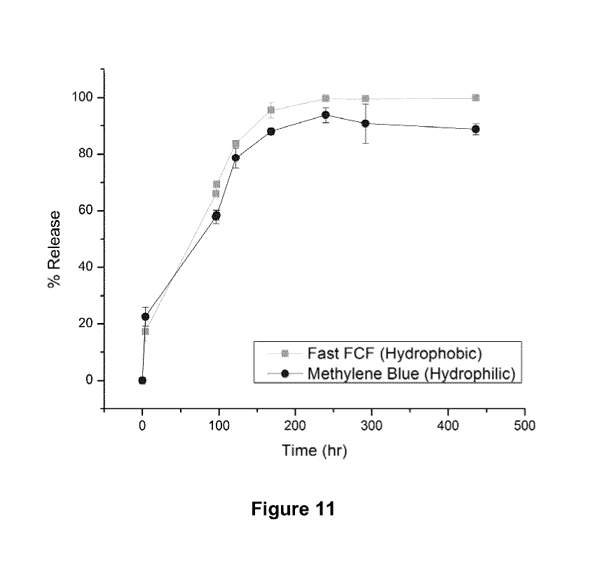

In order to test the encapsulation ability of hydrophilic and hydrophobic molecules, solutions of Fast Green FCF dye and methylene blue dye were prepared at a concentration of 140 mg/mL as well and added to CMC solution at 20 wt %. These solutions were then transferred to a cylindrical mold as mentioned in Example 3 and the hydrogels were synthesized. The hydrogels were then placed in 100 mL of deionized water and the release of the active agents was measured by collecting 1 mL of sample from the release medium. The release medium was constantly replenished to ensure constant volume. The amount of active agent released was quantified using UV-Visible spectroscopy. Absorbance was measured at 630 nm for fertilizer sample, 620 nm for the Fast Green FCF dye and 290 nm for methylene blue sample. It was observed that fertilizer could be released over a month's time, as illustrated in FIG. 10. The dyes were released at a similar rate, as illustrated in FIG. 11.

### Example 8

### Millispheres of DNA-Curdlan by Co-Gelation and Nanoprecipitation

Curdlan was dissolved in 0.4 M aqueous sodium hydroxide at a concentration of 15 mg/mL and DNA was dissolved in deionized water at the same concentration. 5 mixtures of DNA and Curdlan were created by adding various volumes of DNA (2 mL, 1.5 mL, 1 mL, 0.5 mL, 0 mL) and Curdlan (0 mL, 0.5 mL, 1 mL, 1.5 mL, 2 mL respectively) solutions. Then, 0.5 mL of each of these mixtures was added to 5 mL of either 1 wt % or 10 wt % magnetically stirring aqueous calcium chloride solutions in a dropwise manner. These solutions were allowed to stir for an hour. Three millispheres were collected on a microscope slide for imaging purposes from each sample that demonstrated millispheres. These were displayed in FIG. 5. It was observed that higher concentration of DNA (˜50%) and higher concentration of calcium chloride (˜10%) provided more well-defined millispheres. These millispheres were compressed under a cover-slip to be studied under a Zeiss Phase Contrast Optical Microscope. The samples were imaged at 10× magnification to demonstrate the presence of inner core in some of the samples. This was illustrated in FIG. 6.

### Example 9

### Nanofibers Formed by DNA and Curdlan Nanoprecipitation

Curdlan (10 mg/mL and 30 mg/mL) was dissolved in 0.4 M aqueous sodium hydroxide and DNA (0.1 mg/mL, 0.5 mg/mL and 2.5 mg/mL) was dissolved in deionized water. Mixtures of DNA and curdlan were made by adding 0.25 mL of curdlan samples to 0.25 mL DNA samples. These mixtures (0.5 mL) were added to magnetically stirring solution of 10 wt % aqueous calcium chloride in a dropwise manner. TEM samples were prepared by placing a drop of the sample on 300 mesh Formvar coated copper grids (Canemco & Merivac) and then blotting it with filter paper. The sample was stained using a drop of phosphotungstic acid, which was later blotted as well. The samples prepared were analyzed using a Philips CM10 Transmission Electron Microscope. The images captured are presented in FIG. 7. The figure indicated that it is possible to form nano structures with varying morphologies.

### Example 10

### CMC Hydrogel for Growth of Wheat and Canola

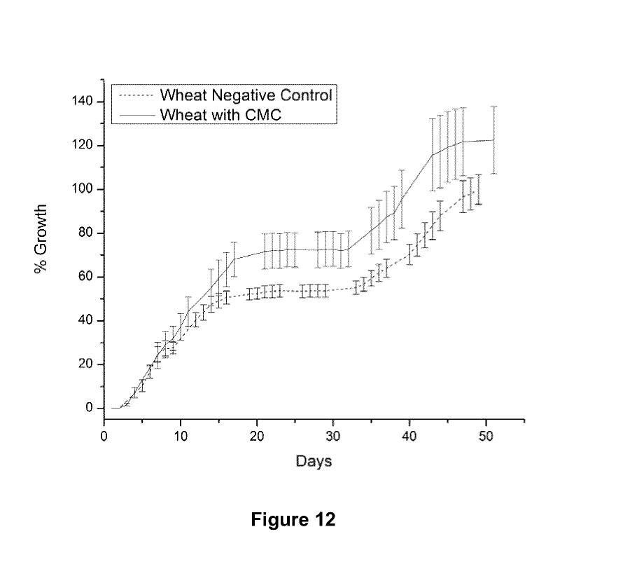

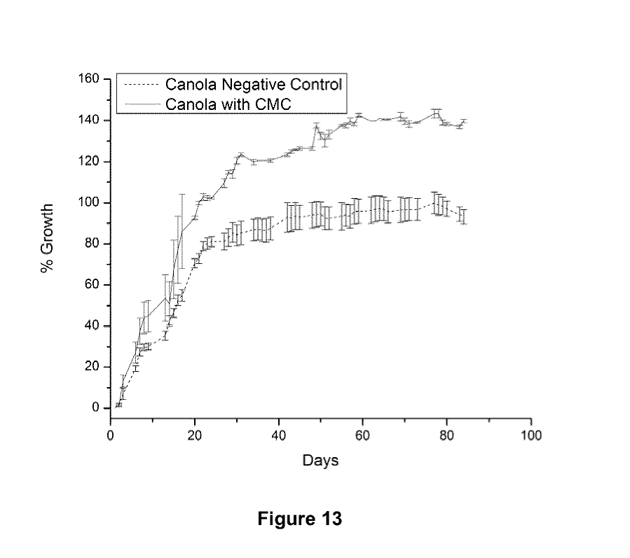

CMC hydrogels with encapsulated fertilizer from Example 7 were implanted along with seeds of wheat and canola in a pot. The experiment was done in triplicates with two seeds per pot. A control experiment was run without the CMC hydrogel. Plant growth was determined by measuring the height of the plants. The maximum height from control experiments was used for normalization of the data and hence growth was presented as a percentage. It was observed that CMC hydrogels with fertilizers had the ability to enhance the growth of both wheat (FIG. 12) and canola (FIG. 13) plants.

### Example 11

### Ionic CMC Hydrogel Cross-Linked Under Heat

CMC hydrogels with encapsulated fertilizer, similar to those from Example 7, were prepared by crosslinking the CMC using a dialysis medium of 1 wt % iron (II) chloride, 1 wt % iron (III) chloride and 10 wt % calcium chloride which was constantly heated to 40° C. for the duration of the cross-linking step in order to increase the diffusion of the salts into the CMC hydrogel.

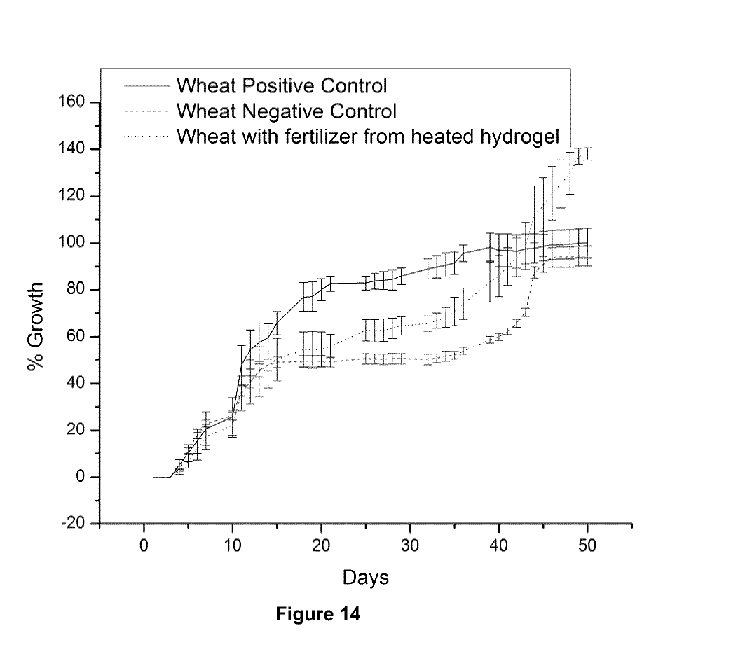

The resulting hydrogels were tested for their ability to increase plant growth. The testing was done in triplicate with one seed per pot. Control experiments were run without the CMC hydrogel, the positive controls receiving daily doses of 50 mL of 1 g/L 20/20/20 fertilizer in DI water and the negative controls receiving only 50 mL DI water. Plant growth was determined by measuring the height of the plants. The maximum height from the positive control experiments was used for normalization of the data and hence growth was presented as a percentage. It was observed that the growth of the plants with the CMC hydrogels was superior to that of the positive control and indicated that the heated cross-linked hydrogels outperformed the regular CMC hydrogel formulation, as determined by per cent increase in growth over the concurrently grown positive controls. The results are shown in FIG. 14.

### Example 12

### Dried Ionic and Chemical Cross-Linked CMC Hydrogels

Ionic CMC hydrogels with encapsulated fertilizer, similar to those from Example 7, were taken after ionic crosslinking and dried by placing them in an oven set to 80° C. for 48 hours in order to remove the residual water and induce crystallization of the hydrogel.

For chemical crosslinking, the hydrogels were prepared by preparing a CMC solution (70 mg/mL) and placing the resulting hydrogel into a mold and placing the hydrogels into a crosslinking solution composed of 250 mL of 25% glutaraldehyde solution in H₂O, 140.2 mL deionized water and 9.8 mL of 38% hydrochloric acid. The hydrogels were allowed to remain in the crosslinking solution under constant heating to 40° C. for 48 hours. The hydrogels were then removed from the crosslinking solution and were repeatedly rinsed with deionized water until all residual glutaraldehyde had been removed from the hydrogel. The hydrogels were then reverse loaded with fertilizer by placing them in a 2.15 g/L solution of 20/20/20 fertilizer and allowing the fertilizer to diffuse into the hydrogel over a period of 48 hours. The chemical cross-linked hydrogels were then loaded into an oven and dried over a period of 48 hours.

After drying the hydrogels they were placed individually into pots with one seed per pot. Control experiments were run without the CMC hydrogel, the positive controls receiving daily doses of 50 mL of 1 g/L 20/20/20 fertilizer in DI water and the negative controls receiving only 50 mL DI water. Plant growth was determined by measuring the height of the plants. The maximum height from control experiments was used for normalization of the data and hence growth was presented as a percentage. It was observed that the growth of the plants with the CMC hydrogels was superior to that of the positive control. The results are shown in FIGS. 15 and 16.

### Example 13

### Effect of Ionic and Chemical Cross-Linked CMC Hydrogel on Plants Under Drought Conditions

Ionic CMC hydrogels with encapsulated fertilizer were created using the procedure from Example 7.

For chemical crosslinking, the hydrogels were prepared by mixing a CMC solution (70 mg/mL) and placing the resulting hydrogel into a mold and placing the hydrogels into a crosslinking solution composed of 250 mL of 25% glutaraldehyde solution in H₂O, 140.2 mL deionized water and 9.8 mL of 38% hydrochloric acid. The hydrogels were allowed to remain in the crosslinking solution under constant heating to 40° C. for 48 hours. The hydrogels were then removed from the crosslinking solution and were repeatedly rinsed with deionized water until all residual glutaraldehyde was removed from the hydrogel.

The hydrogels were placed individually into pots with one seed per pot and were given 50 mL deionized water twice per week in the case of the ionic CMC hydrogels and 50 mL of 1 g/L 20/20/20 fertilizer in DI water twice per week for the chemical CMC hydrogels. Control experiments were run without the CMC hydrogels, the positive controls receiving doses of 50 mL of 1 g/L 20/20/20 fertilizer in DI water twice per week and the negative controls receiving only 50 mL DI water twice per week. Plant growth was determined by measuring the height of the plants. The maximum height from control experiments was used for normalization of the data and hence growth was presented as a percentage. It was observed that the growth of the plants with the CMC hydrogels was superior to that of the positive control. The results are shown in FIGS. 17 and 18.

All references above are expressly incorporated herein in their entirety.

The above-described embodiments are intended to be examples only. Alterations, modifications and variations can be effected to the particular embodiments by those of skill in the art without departing from the scope, which is defined solely by the claims appended hereto.

1. A method of delivering an active agent to a plant, comprising:
administering to the plant an ionically cross-linked hydrogel, the ionically cross-linked hydrogel comprising:
a polysaccharide polymer;
ions interacting with counter-ion functional groups on the polysaccharide polymer and cross-linking the polysaccharide polymer; and
an active agent encapsulated by the ionically cross-linked polysaccharide polymer; and
administering a chelator to chelate at least a portion of the ions that ionically cross-link the hydrogel, triggering release of the encapsulated active agent from the hydrogel, and delivering the active agent to the plant.

2. The method of claim 1 wherein the ions are calcium ions, iron ions, aluminum ions, nickel ions, cobalt ions, copper ions or any combination thereof.

3. The method of claim 1 or 2 wherein chelator is sodium citrate, ethylene-diaminetetraacetic acid (EDTA), a phosphonate or any combination thereof.

4. The method of claim 1, wherein the ions are calcium ions and the chelator is sodium citrate.

5. The method of any one of claims 1 to 4, wherein the active agent is water; a small molecule; an immunostimulator; an anti-cancer molecule; a vaccine; a biopolymer; a crop protecting agent; or any combination thereof.

6. The method of any one of claims 1 to 4 wherein the active agent is water.

7. The method of any one of claims 1 to 4 wherein the active agent is a plant fertilizer.

8. The method of any one of claims 1 to 7 wherein the polysaccharide polymer is a peptidoglycan polymer.

9. The method of any one of claims 1 to 7, wherein the polysaccharide polymer is a beta-glucan polymer or an alpha-glucan polymer.

10. The method of claim 9 wherein the alpha-glucan polymer is an alpha-1,6-glucan with alpha-1,3 branches.

11. The method of claim 10 wherein the alpha-glucan polymer is dextran or polyaldehyde dextran.

12. The method of claim 9 wherein the alpha-glucan polymer is an alpha-1,4-; alpha-1,6-glucan.

13. The method of claim 12 wherein the alpha-glucan polymer is pullulan or starch.

14. The method of claim 9 wherein the polysaccharide polymer is a beta-1,3-glucan or a beta-1,4-glucan polymer.

15. The method of claim 14 wherein the beta-1,3-glucan is a beta-1,3-glucan with beta-1,6 branches.

16. The method of claim 15 wherein the beta-glucan is schizophyllan, lentinan, pachyman, pachymaran, scleroglucan, grifolan or a chemical derivative thereof.

17. The method of claim 16 wherein the chemical derivative is carboxymethylpachymaran, hydroxymethyl pachymaran, or hydroxypropyl pachymaran.

18. The method of claim 14 wherein the beta-1,3-glucan polymer is a curdlan polymer or a carboxymethyl curdlan polymer.

19. The method of claim 14 wherein the beta-1,4-glucan polymer is a cellulose polymer, a carboxymethyl cellulose polymer, chitin or a chitin derivative.

20. A polysaccharide-based polymer hydrogel comprising:
a polysaccharide polymer;
a cross-linker interacting with the polysaccharide polymer to cross-link the polysaccharide polymer; and
an active agent encapsulated by the cross-linked polysaccharide polymer.

21. The polysaccharide-based polymer hydrogel according to claim 20, wherein the cross-linker is an ion that interacts with counter-ion functional groups on the polysaccharide polymer.

22. The polysaccharide-based polymer hydrogel according to claim 21, wherein the ion is a metal cation.

23. The polysaccharide-based polymer hydrogel according to claim 20, wherein the cross-linker is a chemical cross-linker reacted with the polysaccharide polymer.

24. The polysaccharide-based polymer hydrogel according to claim 23 wherein the chemical cross-linker is glutaraldehyde.

25. The polysaccharide-based polymer hydrogel according to any one of claims 20 to 24, wherein the active agent is a small molecule; an immunostimulator; an anti-cancer molecule; a vaccine; a biopolymer; a crop protecting agent; or any combination thereof.

26. The polysaccharide-based polymer hydrogel according to any one of claims 20 to 24, wherein the active agent is a plant fertilizer.

27. The polysaccharide-based polymer hydrogel according to any one of claims 20 to 26 wherein the polysaccharide polymer is a peptidoglycan polymer.

28. The polysaccharide-based polymer hydrogel according to any one of claims 20 to 26, wherein the polysaccharide polymer is a beta-glucan polymer or an alpha-glucan polymer.

29. The polysaccharide-based polymer hydrogel according to claim 28 wherein the alpha-glucan polymer is an alpha-1,6-glucan with alpha-1,3 branches.

30. The polysaccharide-based polymer hydrogel according to claim 29 wherein the alpha-glucan polymer is dextran or polyaldehyde dextran.

31. The polysaccharide-based polymer hydrogel according to claim 28 wherein the alpha-glucan polymer is an alpha-1,4-; alpha-1,6-glucan.

32. The polysaccharide-based polymer hydrogel according to claim 31 wherein the alpha-glucan polymer is pullulan or starch.

33. The polysaccharide-based polymer hydrogel according to claim 28, wherein the polysaccharide polymer is a beta-1,3-glucan or a beta-1,4-glucan polymer.

34. The polysaccharide-based polymer hydrogel according to claim 33 wherein the beta-1,3-glucan is a beta-1,3-glucan with beta-1,6 branches.

35. The polysaccharide-based polymer hydrogel according to claim 34 wherein the beta-glucan is schizophyllan, lentinan, pachyman, pachymaran, scleroglucan, grifolan or a chemical derivative thereof.

36. The polysaccharide-based polymer hydrogel according to claim 35 wherein the chemical derivative is carboxymethylpachymaran, hydroxymethyl pachymaran, or hydroxypropyl pachymaran.

37. The polysaccharide-based polymer hydrogel according to claim 33, wherein the beta-1,3-glucan polymer is a curdlan polymer or a carboxymethyl curdlan polymer.

38. The polysaccharide-based polymer hydrogel according to claim 33 wherein the beta-1,4-glucan polymer is a cellulose polymer, a carboxymethyl cellulose polymer, chitin or a chitin derivative.

39. The polysaccharide-based polymer hydrogel according to any one of claims 20 to 38, further comprising an excipient.

40. The polysaccharide-based polymer hydrogel according to claim 39, wherein the excipient is a bulking agent.

41. An ionically cross-linked polysaccharide-based polymer hydrogel for triggered delivery of an active agent encapsulated by the hydrogel; the hydrogel comprising:
a polysaccharide polymer;
ions interacting with counter-ion functional groups on the polysaccharide polymer to cross-link the polysaccharide polymer; and
an active agent encapsulated by the cross-linked polysaccharide polymer.

42. The ionically cross-linked polysaccharide-based polymer hydrogel according to claim 41 wherein the delivery of the encapsulated agent is triggered by a chelating agent that interacts with at least a portion of the ions and preventing them from cross-linking the polysaccharide polymer.

43. The ionically cross-linked polysaccharide-based polymer hydrogel according to claim 41 or 42, wherein the active agent is water; a small molecule; an immunostimulator; an anti-cancer molecule; a vaccine; a biopolymer; a crop protecting agent; or any combination thereof.

44. The ionically cross-linked polysaccharide-based polymer hydrogel according to claim 41 or 42, wherein the active agent is water.

45. The ionically cross-linked polysaccharide-based polymer hydrogel according to claim 41 or 42, wherein the active agent is a plant fertilizer.

46. The ionically cross-linked polysaccharide-based polymer hydrogel according to any one of claims 41 to 45 wherein the polysaccharide polymer is a peptidoglycan polymer.

47. The ionically cross-linked polysaccharide-based polymer hydrogel according to any one of claims 41 to 45, wherein the polysaccharide polymer is a beta-glucan polymer or an alpha-glucan polymer.

48. The ionically cross-linked polysaccharide-based polymer hydrogel according to claim 47 wherein the alpha-glucan polymer is an alpha-1,6-glucan with alpha-1,3 branches.

49. The ionically cross-linked polysaccharide-based polymer hydrogel according to claim 48 wherein the alpha-glucan polymer is dextran or polyaldehyde dextran.

50. The ionically cross-linked polysaccharide-based polymer hydrogel according to claim 47 wherein the alpha-glucan polymer is an alpha-1,4-; alpha-1,6-glucan.

51. The ionically cross-linked polysaccharide-based polymer hydrogel according to claim 50 wherein the alpha-glucan polymer is pullulan or starch.

52. The ionically cross-linked polysaccharide-based polymer hydrogel according to claim 47, wherein the polysaccharide polymer is a beta-1,3-glucan or a beta-1,4-glucan polymer.

53. The ionically cross-linked polysaccharide-based polymer hydrogel according to claim 52 wherein the beta-1,3-glucan is a beta-1,3-glucan with beta-1,6 branches.

54. The ionically cross-linked polysaccharide-based polymer hydrogel according to claim 53 wherein the beta-glucan is schizophyllan, lentinan, pachyman, pachymaran, scleroglucan, grifolan or a chemical derivative thereof.

55. The ionically cross-linked polysaccharide-based polymer hydrogel according to claim 54 wherein the chemical derivative is carboxymethylpachymaran, hydroxymethyl pachymaran, or hydroxypropyl pachymaran.

56. The ionically cross-linked polysaccharide-based polymer hydrogel according to claim 52, wherein the beta-1,3-glucan polymer is a curdlan polymer or a carboxymethyl curdlan polymer.

57. The ionically cross-linked polysaccharide-based polymer hydrogel according to claim 52 wherein the beta-1,4-glucan polymer is a cellulose polymer, a carboxymethyl cellulose polymer, chitin or a chitin derivative.

58. A kit for releasing an active agent from an ionically cross-linked polysaccharide-based polymer hydrogel that encapsulates the active agent, the kit comprising:
the ionically cross-linked polysaccharide-based polymer hydrogel that encapsulates the active agent; and
a chelating agent adapted to chelate at least a portion of the ions that ionically cross-link the polysaccharide-based polymer hydrogel.

59. A method of preparing a polysaccharide-based polymer hydrogel for delivery of an active agent, comprising:
providing a polysaccharide polymer;
providing a solution that includes the active agent;
dispersing or dissolving the polysaccharide polymer in the solution to form a polymer gel solution; and
cross-linking the polysaccharide polymer in the polymer gel solution with a cross-linker to form the polysaccharide-based polymer hydrogel which encapsulates the active agent.

60. The method of claim 59, wherein the cross-linker is an ion and the polysaccharide-based polymer hydrogel is an ionically cross-linked hydrogel, the method comprising contacting the polymer gel solution with the ion to crosslink the polysaccharide polymer and form the ionically cross-linked polysaccharide-based polymer hydrogel.

61. The method of claim 60, wherein the ion is a metal ion.

62. The method of claim 59, wherein the metal ion is calcium, iron, aluminum, nickel, cobalt or copper.

63. The method of any one of claims 59 to 62, wherein the active agent is water.

64. The method of claim 63, wherein the active agent additionally comprises a crop protecting agent.

65. The method of claim 64, wherein the crop protection agent is a salt, ion, mineral, fertilizer, nematicide, pesticide, herbicide, insecticide, essential nutrient, non-essential nutrient, nucleic acid, fungicide, or any combination thereof.

66. The method of claim 65, wherein the crop protection agent is a nucleic acid.

67. The method of claim 66, wherein the nucleic acid is dispersed in deionized water prior to being added to the polymer solution.

68. The method of claim 66, wherein the active agent additionally comprises a plant fertilizer.

69. The method of any one of claims 59 to 68, further comprising drying the hydrogel.

70. A method of delivering an active agent to a plant, comprising administering to the plant a hydrogel according to any one of claims 20 to 57.

71. The method of claim 70, wherein the cross-linker is a chemical cross-linker and the polysaccharide-based polymer hydrogel is a chemically cross-linked hydrogel, and the method comprises allowing the active agent to diffuse out of the hydrogel.

72. The method of claim 70, wherein the hydrogel further comprises an excipient, the active agent is a crop protecting agent, and the method comprises osmotic pressure driven release of the active agent from the hydrogel.
